(19) United States
(12) Patent Application Publication
(10) Pub. No.: US 2006/0121077 A1
(43) Pub. Date: Jun. 8, 2006

(54) Method for treating patients with implants of islets which have been kept in long term, in vitro culture

(76) Inventors: Lawrence Gazda, Xenia, OH (US); Barry Smith, New York, NY (US); Albert Rubin, New York, NY (US)

Correspondence Address: FULBRIGHT & JAWORSKI, LLP, 666 FIFTH AVE, NEW YORK, NY, 10103-3198, US

(21) Appl. No.: 11/269,281

(22) Filed: Nov. 8, 2005

Related U.S. Application Data
(60) Provisional application No. 60/626,970, filed on Nov. 11, 2004.

(51) Int. Cl. A61F 13/00; A61K 35/39

(52) U.S. Cl. 424/422; 424/93.7

(57) ABSTRACT

The invention relates to the use of macrobeads which have been kept in in vitro storage for long term culture, i.e., for at least about 8 months. The macrobeads surprisingly exhibit continued efficacy in producing therapeutically useful materials. The fact that the macrobeads can be used after long term culture allows the artisan to screen the products so as to make sure that, in addition to being therapeutically useful, they remain safe as implants.

### RELATED APPLICATIONS

This application claims priority of Ser. No. 60/626,970, filed Nov. 11, 2004, incorporated by reference in its entirety.

### FIELD OF THE INVENTION

The invention relates to the use of macrobeads, which have been held in long term, in vitro storage, as therapeutic agents. The macrobeads, which are preferably made of agarose, and coated with agarose, contain cells which produce a therapeutic agent of interest, which passes through the macrobead in order to produce a positive therapeutic effect. Preferably, the cells are secretory cells such as islets which produce insulin, but they can be any type of cell which inherently produces a therapeutic agent of interest, or a type of cell which produces a therapeutic product under conditions of entrapment and encapsulation.

### BACKGROUND AND PRIOR ART

Therapeutic approaches to insulin related disorders, such as insulin-dependent diabetes mellitus (IDDM), are always of great interest. The occurrence of these disorders in the population at large is growing, and as such the need for new therapies is growing as well.

Jain, et al., in Transplantation, 59(3):319-24 (1995), Transplantation 61(4):532-536 (1996), Transplantation 68(11):1693-1700 (1999), U.S. Pat. No. 5,643,569 and Re 38,027 have proposed a useful, workable model for the treatment of these disorders. The references cited supra, which are incorporated by reference will detail this model. In brief, however, islets are secured from a donor animal, which may be of murine, porcine, bovine, ovine, or primate, such as human origin, as well as from other animal species, and are encapsulated in a permeable, semi-solid structure, such as a bead, or more correctly, a macrobead. “Macrobead,” as used throughout this disclosure, refers to permeable structures measuring approximately 4-12 mm more preferably approximately 4-10 mm, and even more preferably, 6-8 mm at their greatest diameter, which may contain secretory cells, or collections of secretory cells, such as islets, of various types, in numbers ranging up to hundreds or even many thousands. The structures may consist of agarose, as is preferred, or may be a mix of agarose and collagen, or some other material. After a semi-solid bead is prepared, it is coated with agarose. The resulting structures can be implanted into a subject in need of insulin therapy. The permeable nature of the structure permits insulin as well as other molecules that regulate cell structure and function to exit therefrom, as well as cellular waste products, while also permitting ingress of nutrient materials. As a result of this permeability, the structures, if kept under proper conditions, maintain viability, i.e., live, insulin producing encapsulated cells, for indeterminate periods of time. This feature is important to the invention, as will be seen, infra.

“Islet transplantation therapy,” which is the field of this invention, generally requires implantation of structures containing allogeneic cells. This type of transplantation, in turn, requires immunosuppression of the recipient, in order to control allo- and autoimmune responses. The use of standard, immunosuppressive protocols has been shown to permit insulin dependence, in the majority of islet transplanted diabetic patients. See, Shapiro, et al., N. Engl. J. Med., 343:230-238 (2000); Ryan, et al., Diabetes, 50:710-719 (2001); Goss, et al., Transplantation, 74:1761-1766 (2002); Ricardi, et al., Transplantation, 75:1524-1527 (2003).

In contrast to established clinical immunosuppressive therapy for allogeneic islet grafting, there are no standard protocols for xenogeneic islet transplantation. While innovative protocols for immunosuppression are topics of intense scrutiny (Jonker, et al., Transplant Proc., 33:726 (2001); Song, et al., Xenotransplantation, 10:628-634 (2003)), none have been established for clinical use. Other approaches which may facilitate xenogeneic islet grafting include tolerance induction (Rayat, et al., Curr. Diab. Rep., 3:336-343 (2003)), gene therapy (Grannoukakis, et al., Diabetes Nutr. Metab., 15:173-203 (2002)) and use of tissue from genetically modified donor animals (Prather, et al., Reprod. Biol. Endocrin., 1:82 (2003)).

The encapsulation of xenogeneic islets in the structures discussed supra avoids the issues of immunosuppression, Jain, et al., in three papers (Transplantation, 59:319-324 (1995); Transplantation, 61:532-536 (1996); and Transplantation 68:1693-1700 (1999)), all of which are incorporated by reference, reported how rat and porcine islets, encapsulated as described supra, resulted in maintenance of normoglycemia, in xenogeneic animal models of IDDM, without the need to address immunosuppression issues.

The work reported in these papers has now been expanded. It has now been learned that encapsulated islets, maintained in culture for more than about 8 months, retain the ability to secrete materials, such as proteins, including insulin, after transplantation to diabetic subjects and patients, without the need for immunosuppressive therapy.

The support for the invention and ramifications thereof will be seen in the disclosure which now follows.

### DETAILED DESCRIPTION OF PREFERRED EMBODIMENTS

### EXAMPLE 1

This example describes the isolation of porcine islets, followed by the preparation of macrobeads.

Donor animals were sows, more than two years of age, which had a history of multiple parities. Animals were humanely sacrificed, pancreata were retrieved, and transported to laboratories, under standard conditions.

The islets were then isolated in accordance with Jain, et al., Transplantation, 68:1693-1700 (1999), incorporated by reference. In brief, however, the fat and connective tissue were removed from the gland via trimming, and the main pancreatic duct was cannulated, and injected with Hanks Balanced Salt Solution (HBSS), augmented with collagenase V at 1.8 g/l, protamine, at 0.06 g/l, and NaOH, at about 100 μ/l. A total amount of solution equal to 4 times the weight of the pancreas was perfused through the duct, at 150 ml/min, at 18° C.

Islets were then purified, on discontinuous gradients of densities 1.105 g/cm³, 1.095 g/cm³, and 1.055 g/cm³, in 50 ml polystyrene conical tubes. These tubes were then centrifuged, at 2000 rpm, and islet containing layers were manually collected, and then washed, three times, in a mixture of HBSS and 2% porcine serum. They were then counted on an electronic Coulter cell counter.

In the experiments which follow, islets counts were expressed as “equivalent islet numbers,” or “EIN,” based upon a standard islet size of 150 μm, and 500 EIN per macrobead.

Encapsulation in agarose, followed by agarose coating, in accordance with Jain, et al., supra was used to form the macrobeads. Once the macrobeads were made, they were maintained in RPMI, containing 2% porcine serum, and 1% antibiotic/antimyotic, at an atmosphere of 5% CO₂ and air, at 36-38° C.

### EXAMPLE 2

These experiments describe the testing of the macrobeads and culture medium in which they were kept, for any foreign materials.

Samples of both the macrobeads and the culture medium were tested. With respect to the macrobeads these were crushed, aseptically, and filtered, after washing with USP Fluid A. The resulting filter was cut in half, and transferred to a soybean/casein digest media with fluid thioglycollate medium, followed by 14 days of incubation. The cultures were then examined for growth in the cultures.

Further testing was carried out in a second set of experiments, including porcine virology testing, such as screening for porcine reproductive and respiratory syndrome, swine influenza virus, porcine endogenous virus, porcine enterovirus, porcine respiratory corona virus, and transmissible gastroenteritis virus, all by RT-PCR.

Porcine circovirus types 1 and 2, porcine lymphotropic herpes virus type 1, porcine parvovirus, porcine cytomegalovirus, swine hepatitis E virus, and M. hyponeuminiae were tested via PCR. Pseudorabies virus was tested for via viral isolation, as was encephalomyocarditis virus. Rotavirus and Chlamydiae were measured via ELISA.

In all cases, standard methods were used. Samples were taken from individual lots of the macrobeads, and were found to be negative for growth of bacteria and fungus over a 7 day observation period. Control macrobeads, which were prepared in the same way but contained no islets, were also negative for any growth.

Random selections of macrobeads and culture media were also screened for viruses and microbiological agents as described supra. All samples were negative, for all agents tested.

### EXAMPLE 3

These experiments describe in vivo work using the macrobeads described in the two prior examples. The model employed herein is an accepted animal model for human therapy.

A total of 24 male, spontaneously diabetic BB rats were used. The animals had exhibited evidence of clinical diabetes for 3-16 days.

The rats were divided into two groups of twelve, constituting two studies. In the first group, the 12 rats were divided into 2 groups of 6, with one group receiving islet containing macrobeads, and the other group, empty macrobeads.

In the second group, the rats were divided into 3 groups of 4 rats each, and received islet-containing macrobeads that had been cultured, in vitro, for 9, 40, or 67 weeks. The rats all received protamine-zinc insulin prior to the implantation work. The rats which received the empty beads received it after implantation as well.

The macrobeads were examined for uniformity, and collected one day prior to implant. Macrobeads were aliquotted to 175 ml conical tubes, at a maximum of 400 macrobeads per tube, and were stored, overnight at room temperature (ranging from 19-27° C.) in RPMI plus 1% A/A.

Immediately before implant, the macrobeads were washed, three times, with RPMI and 1% A/A.

The animals all received an implant, 20-21 days after receipt, of either islet macrobeads at a dose equivalent to 1.0× the daily insulin requirement, or a comparable number of empty macrobeads. The determination of these values, i.e., the daily insulin requirement and the number of islet containing macrobeads, is routinely obtained. There are well-known guidelines and ranges for how much insulin is necessary and required for regulating glucose levels within each animal species. Optimizing the amount of insulin is determined simply by administering varying doses of insulin to individual subject animals over time, until regulation of glucose levels is attained. Once the amount of insulin that is necessary to regulate levels is known, then the amount of insulin produced by a given culture of islet macrob eads is determined, and the requisite number of macrobeads is used as the implant for that animal or a comparable number of empty macrobeads. The implants were placed gently into the peritoneal cavities of each animal, using a sterile plastic spoon, and incisions were closed.

In study 1, anywhere from 42-56 macrobeads were implanted in the animals, and the total weight of the beads ranged from 10.4-14.8 grams. On average, the islet containing macrobeads were 24.7 weeks old when implanted and empty beads were 19.7 weeks old. The six rats in this study which had received islet containing macrobeads received no insulin for 97 days.

Two days after they had received the empty macrobeads, the control rats exhibited rising glucose levels (from 300-500 mg/dl), and begun receiving exogenous insulin therapy. Two of the control rats died on the third day of the study. Examination revealed no gross abnormalities at necroscopy, leading to the conclusion that insulin deficiency was the cause of death.

Over the course of the study, both daily blood glucose levels, and insulin requirements were determined, for all animals in the study, using standard methods well known in the art, which need not be reiterated here.

All six animals which had received islet transplants exhibited a narrow and controlled range of blood glucose throughout, notwithstanding the complete absence of insulin therapy. When animals became moderately hyperglycemic, daily variations of only about 100 mg/dl were observed.

In contrast, the control animals exhibited extreme variation, of about 400-500 mg/dl, notwithstanding the administration of exogenous insulin throughout the study.

The degree of blood glucose control for all animals was determined by calculating an average deviation from a weekly mean glucose level value.

Blood glucose levels gradually increased during the study period, so weekly averages were selected in order to provide more accurate assessment of control for defined periods.

The results showed that, notwithstanding daily insulin therapy, rats which had received the empty macrobeads exhibited widely fluctuating glucose values, with an average weekly deviation of 127±26 mg blood glucose post implantation. This is nearly identical to the 124.8±25.0 mg/dl observed prior to the implants.

In contrast, the animals which had received the islet implants maintained consistent levels, with an average weekly deviation of 61.3±16.6 mg blood glucose post implant, as compared to 153.6±18.9 mg/dl, pre-implant.

### EXAMPLE 4

There were various assays carried out to determine how subject animals responded to various challenges. For example, before the experiments were begun, all animals received standard, intraperitoneal glucose tolerance tests to confirm the initial diagnosis of IDDM. This test was repeated 8 days after implantation and then again on day 90 post implantation, in order to determine the ability of the animal to respond to glucose challenge.

Prior to the study, notwithstanding their receiving exogenous insulin therapy, 11 of 12 rats were unable to achieve normoglycemia during the pre-implantation challenges, and 9 of the 12 exhibited blood glucose readings above 600 mg/dl during the procedure.

On day eight following implant, the treated rats demonstrated improved blood glucose regulation during a challenge. Two of the rats which had received the islet implants returned to normoglycemia, while the four other rats which had received the islets showed responsiveness (i.e., an increase in blood glucose, followed by a decrease) by the end of the test. In contrast, three of the four empty macrobead implanted rats could not control hyperglycemia during the challenge, in spite of concurrent insulin therapy.

None of the animals who received islet therapy were able to achieve normoglycemia at 90 days post implant. At this point in time, the animals were all moderately hyperglycemic. When challenged, with glucose, blood sugar levels rose and then returned to average, or starting values, which were moderately glycemic. The rats which received empty macrobeads, did not re-establish baseline glycemia.

In a second series of tests, the blood of the animals was tested, for porcine C-peptide using standard methodologies. The reason for this is that porcine C-peptide is cleaved from insulin as insulin leaves islets. Exogenously administered insulin does not contain the peptide. Since different forms of C-peptide can be differentiated, an assay for porcine C-peptide is a routine way to measure insulin production from porcine islets.

The peptide was not detected in any animal prior to implantation, or from any animal which received empty macrobeads. It was routinely found in the serum of rats which received the islet macrobeads, with average level ranging from 0.880±0.249 ng/ml, 21 days post implantation and 0.662±0.160 ng/ml at the end (5 rats).

The overall clinical findings for rats which received the islet containing macrobeads in this first study were better than those which received the empty macrobeads. All of the rats gained weight with no significant difference between the two groups (341.5±24 g, versus 353.3±38.4 g, final average weight, for islet recipients and controls, respectively). Prior to transplantation, there were no significant differences between the two groups with respect to frequency of glycosuria (73% v. 85%), ketonuria (19% v. 28%), or need for administration of bicarbonate (16% v. 23%). Following transplantation, however, the numbers diverged dramatically, with a difference of 65% versus 82% for glycosuria, 3% versus 23% for ketonuria, and 1% versus 13% for bicarbonate therapy, for islet recipients and controls, respectively.

### EXAMPLE 5

At the termination of the study, rats were sacrificed, and macrobeads were retrieved and cultured, in vitro, in accordance with standard techniques. Insulin, glucagon and porcine C-peptide were all detected in the culture medium for at least 9 weeks. Insulin production was lowest at the first week of culture (20.0±13.4 mU/macrobead), and highest during week 7 (35.2±9.0 mU/macrobead). The secretion of porcine C-peptide and glucagon paralleled this (0.3±0.16 ug/macrobead versus 0.45±0.1 μg/macrobead, and 4.0±1.4 ng/macrobead versus 18.0±4.8 ng/macrobead, respectively).

Complete necropsies were performed on all of the animals, 97 days following implantation. Over 90% of the macrobeads were free floating in the peritoneal cavity, and no broken beads were found. One animal exhibited a small, proliferative mass of 1×2.5×0.2 cm, and no gross lesions were found. The only unusual finding was a statistically significant difference in mean liver weight, but both values were within normal ranges for standard laboratory animals.

Viable islet cells were found in the retrieved macrobeads from the animals who received porcine islet macrobeads, as was cellular debris. There were occasional, mononuclear cells, and small inflammatory tags of fibrosis connective tissue on some empty and some islet containing macrobeads. No differences in incidence or severity of these changes were seen, vis a vis the two groups.

At the study termination, samples were taken from the animals and screened for presence of pathogens, as described supra. So, too were the macrobeads. Fecal samples from two animals which received the islet macrobeads were positive for rotavirus and islet containing macrobeads retrieved from all six animals were positive for PERV. The PERV findings were expected, since all swine contain the endogenous virus in their genome.

### EXAMPLE 6

As was pointed out, supra, two studies were carried out, the first of which was described in examples 3-5. This and the following examples, discuss study 2.

As was explained in example 3, the twelve rats were divided into groups containing 4 rats each. The groups all received islet containing macrobeads, which had been cultured, in vitro, for 9 (9.4 average), 40 (40.5 average) or 67 (66.8 average) weeks.

After insulin requirements of the individual animals, and insulin production of the islet macrobeads were determined, just as in study 1, the rats in study 2 received 56-60 islet macrobeads, weighing 12.8-18.2 g.

Implantation was carried out as described supra. The study was carried out for 201 days.

After implantation, normoglycemia was restored for about one month, in all rats in all groups, after which the rats developed moderate hyperglycemia, which persisted throughout the study.

As with the study 1 animals, there was a contemporaneous development of moderate hyperglycemia and attainment of maximal body weight. This in turn, results in increased insulin requirements.

To compensate for this, 97 days into the study (i.e., the day at which the first study animals were sacrificed), the study 2 animals received a second implant of 21 additional islet macrobeads each, weighing 4.8-6.2 g. Immediately before this second implant, 4 of the original islet macrobeads were removed for histopathological analysis.

The second implants did not significantly affect daily blood glucose levels.

The study 2 rats received five separate glucose challenges.

The challenge involved the intraperitoneal injection of 2.0 g dextrose per kg of body weight, at days 11, 43, 85, and 200, post implantation.

No differences were observed with respect to the ability of macrobeads that had been cultured for different lengths of time to respond to glucose challenge, following implantation. Indeed, eleven days following the initial implantation, the starting blood glucose levels of 100-200 mg/dl doubled, following the administration of glucose. A return to baseline glycemia occurred within 20 minutes for 10 of the animals, while two animals which received the 40 week old beads did not. One had a starting level of 118 mg/dl, and a 120 minute reading of 137 mg/dl, while the numbers for the second animal were 171 and 238 mg/dl, respectively. The islet macrobeads maintained their ability to respond to glucose challenge throughout the study, and while all animals did become moderately glycemic, additional glucose challenge procedures demonstrated the initial rise in blood sugar, and then a return to baseline glycemia.

In connection with these studies, the “Area Under the Curve” with respect to “ground,” as described by Pruessner, et al., Psychoneuroendocrinology, 28:916-931 (2003), incorporated by reference, was calculated for all 3 groups. The average values on day 11 following implant, were 27-30,000 min. mg/dl of all groups, and the values doubled subsequently, with no significant differences therebetween. Nor did the reimplant have any impact.

As with the study 1 animals, porcine C-peptide levels were measured, throughout the experiment (seven different times, via serum, plus peritoneal fluid at necropsy. The peptide was detected in all groups, with a decrease in average (0.6-0.9 ng/ml, down to 0.2-0.4 ng/dl), occurring during the first 88 days, across the groups. There was an increase observed, at day 116, which followed the re-implant. Throughout the rest of the study, the observed levels ranged from 0.3-0.7 ng/ml, with a 40-fold increase in peritoneal fluid at necroscopy.

### EXAMPLE 7

When the study 2 rats were sacrificed, a total of 25 porcine islet macrobeads per rat were retrieved, and cultured as they were before implantation. They continued to produce insulin, porcine C-peptide and glucagon, and no observable difference in hormonal production between the variously aged macrobeads was seen over the 2-month period of observation.

When the study 2 animals were necropsied (201-202 days after the start of the experiment), more than 90% of the islet macrobeads remained floating in the peritoneal cavity. No broken macrobeads were found. Two islet macrobeads, in one animal, were found to have a fibrous connection to the peritoneum, and an occasional macrobead was found lodged between the lobes of the liver. There were no significant differences observed in any of the organs.

With the exception of moderate inflammation associated with pancreatic islets, expected in the animal model used, only minimal to mild pathology was reported, with no clear differences between the groups. The inflammation, fibrosis, or hypertrophy/hyperplasia of the peritoneum covering the pancreas, abdominal wall, and/or diaphragm, was believed to be related to the presence of the beads. The minimal or mild severity, localized in examined tissue, was not believed to have altered normal tissue function.

Fibrosis, both with and without inflammatory cells, was present on the surface of some macrobeads collected at day 97 and at necroscopy. Again, the inflammation was minimal to mild. The collected macrobeads, at both time points, contained viable islet cells, but the areas of cellular debris exceeded the number of islet clusters. No difference in the macrobeads was observed amongst the groups, or between the macrobeads collected at day 97 or at necropsy.

The foregoing examples set forth aspects of the invention, which relate to the use of macrobeads, as defined herein, which have been subjected to long term, in vitro storage prior to their use as therapeutic agents.

The ability to use macrobeads which have been subject to long term storage is surprising because prior work on similar materials suggests that, following long term storage, the cells would lose their ability to produce relevant therapeutic materials. As it is always desirable to be able to rely on a “bank” of stored materials to carry out a desired therapeutic end, the invention as described herein, expands the therapeutic options available with the macrobeads, which are themselves known.

“Long term” in vitro storage, as used herein, refers to macrobeads which have been stored, in vitro for at least about 8 months, preferably at least 10 months. Macrobeads stored for at least about 8, 10, 12 months in vitro, or even longer, as can be seen from the examples, can all be used, and still exert a useful therapeutic effect.

The examples presented herein show the use of agarose macrobeads, coated with agarose, which contain islets. Such macrobeads are described by, e.g., the U.S. Patents cited supra. These patents ascribe other types of macrobeads, which can also be used, such as beads made of agarose and collagen, which are also coated with agarose. Other cell types can be placed in the macrobeads, such as stem cells, or other cells which are known to secrete therapeutic agents, as well as cells which, when entrapped or encapsulated, produce factors which they would not normally produce, or which they produce in increased amounts, and which result in therapeutic impact. Exemplary of such cells are cancer cells, as are described in, e.g., U.S. Pat. No. 5,888,497, which is incorporated by reference. In addition to these disclosures, other forms of macrobeads, such as, but not being limited to alginate beads, or beads which share properties of agarose may be used.

The ability to use macrobeads that have been stored in long term in vitro culture permits, inter alia, the assessment of the materials for microbiological safety.

The method involves the use of macrobeads which contain useful cells. These cells may be taken from the patient himself, i.e., autologous cells, may be from the same species as the subject or may even be taken from a species different from the patient or subject, as can be seen in the examples, where diabetic rats were treated with beads containing porcine islets. Human islets, primate islets, porcine islets, as well as rodent or other islets may all be used in the practice of the invention. Other types of cells from various species may also be used.

Other aspects of the invention will be clear to the skilled artisan, and need not be reiterated here.

The terms and expression which have been employed are used as terms of description and not of limitation, and there is no intention in the use of such terms and expression of excluding any equivalents of the features shown and described or portions thereof, it being recognized that various modifications are possible within the scope of the invention.

We claim

1. A method for treating a pathological condition comprising implanting in a subject with said pathological condition a quantity of macrobeads which contain, encapsulated or entrapped therein, cells which produce a material which leaves said macrobead and alleviates said pathological condition, sufficient to alleviate said condition, wherein said macrobeads have been cultured in vitro for at least about 8 months.

2. The method of claim 1, wherein said macrobeads have been cultured in vitro for at least about 10 months.

3. The method of claim 1, wherein said macrobeads have been cultured in vitro for at least about 12 months.

4. The method of claim 1, wherein said macrobeads have been cultured in vitro for more than about 12 months.

5. The method of claim 1, wherein said macrobeads are made of agarose and are coated with agarose.

6. The method of claim 1, wherein said macrobeads contain secretory cells.

7. The method of claim 6, wherein said secretory cells are islets.

8. The method of claim 1, wherein said cells are cancer cells.

9. The method of claim 1, wherein said cells are stem cells.

10. The method of claim 1, wherein said cells are from the subject receiving implantation of said macrobeads.

11. The method of claim 1, wherein said cells are from the same species as the subject receiving implantation.

12. The method of claim 1, wherein said cells are from a different species from the subject receiving implantation.

13. The method of claim 1, wherein said subject is a human being.

14. The method of claim 1, wherein said subject is a non-human animal.

15. The method of claim 1, comprising repeating said implantation at least one additional time.
